(19) United States
(12) Patent Application Publication
(10) Pub. No.: US 2022/0211789 A1
(43) Pub. Date: Jul. 7, 2022

(54) EXTRACTION OF CANNABINOIDS FROM BIOMASS

(71) Applicant: Becanex GmbH, Berlin (DE)

(72) Inventors: Mazin NICOLA, Wageningen (NL); Eral OSMANOGLOU, Wageningen (NL); Sander HUURMAN, Wageningen (NL)

(21) Appl. No.: 17/609,699

(22) PCT Filed: May 7, 2020
(86) PCT No.: PCT/EP2020/062698

§ 371 Date: Nov. 8, 2021

(30) Foreign Application Priority Data
May 10, 2019 (GB) 1906598.6

(51) Int. Cl. A61K 36/185; A61K 31/05; A61K 31/352

(52) U.S. Cl. CPC A61K 36/185; A61K 31/352; A61K 31/05

(57) ABSTRACT

A method of extracting at least one cannabinoid from a biomass consisting of industrial hemp comprises: (i) contacting the biomass with a solvent formulation which comprises a C1-4 fluorinated hydrocarbon or a C1-4 hydrofluorocarbon ether, thereby to charge the solvent formulation with an extract from the biomass; and (ii) separating charged solvent formulation from the biomass.

This invention relates to extraction, for example of dietary ingredients and/or of dietary and health supplements. The invention also relates to preparation of botanical drug substances. Particularly, although not exclusively, the invention relates to preparation of extracts, for example of dietary and health supplements and BDSs which comprise bio-cannabinoids. Preferred extracts are rich in cannabidiol (CBD), or its acid form, cannabidiolic acid (CBDA).

In general terms, a “dietary ingredient” described may comprise a concentrate, metabolite, extract, or combination of any of the following: (A) a vitamin; (B) a mineral; (C) a herb or other botanical; (D) an amino acid; (E) a dietary substance.

A “dietary supplement” may comprise a product containing, as an ingredient, one or more vitamins, herbs, enzymes, amino acids, or other ingredients, that is taken orally to supplement one's diet by providing a missing nutrient.

A “health supplement” may comprise a diverse group of products commonly consumed for the purpose of supplementing the diet and enhancing health.

The products referred to typically contain ingredients from natural sources; and are not meant to prevent, treat, cure or alleviate the symptoms of medical diseases or conditions

A Botanical Drug Substance (BDS) is described in the Botanical Drug Development Guidance for Industry published in December 2016 by U.S. Department of Health and Human Services Food and Drug Administration “Centre for Drug Evaluation and Research” (CDER) as: “A botanical product intended for use in diagnosing, curing, mitigating, or treating disease. It is derived from one or more plants, algae, or microscopic fungi. It is prepared from botanical raw materials by one or more of the following processes: grinding, decoction, expression, aqueous extraction, ethanolic extraction or other similar processes.” The defined criteria excludes, among other things: “Highly purified substances, either derived from a naturally occurring source or chemically modified”. BDSs can often be marketed under an over-the-counter (OTC) drug monograph and may be available as (but not limited to) a solution (e.g. tea), powder, tablet, capsule, elixir, topical application or injection.

Cannabis plants have been used to provide dietary ingredients, dietary supplements and health supplements, as well as therapeutic substances for many years. A BDS obtained from the Cannabis plant does not have to comply with strictly defined specifications of purity as one single compound such as tetrahydocannabinol (THC) or cannabidiol (CBD). A Cannabis based BDS may be rich in a single compound and/or may include minor impurities, such as non-cannabinoid impurities. Alternatively, a Cannabis based BDS may comprise a mixture of two or more compounds that have synergistic pharmaceutical activity including, but not limited to, THC, CBD, tetrahydrocannabivarin (THCV), Cannabidivarin (CBDV), cannabichromene (CBC) cannabigerol, cannabicyclol, cannabielsol. Furthermore, a Cannabis-based dietary or health supplement or BDS may include non-cannabinoid compounds such as flavour and aroma terpenes and terpenoids.

Published methods for preparation of cannabinoids from the Cannabis plant are multi step consisting of solvent extraction followed by isolation of crude intermediates followed by one or more purification steps. The extraction step may be carried out using an organic solvent such as hexane, ethyl acetate, methanol or ethanol. The solvent is then removed by vacuum aided evaporation at elevated temperatures and a crude intermediate extract is isolated. In general terms, in known extraction processes, a mass of material is subjected to solvent extraction in a first step to produce a first or primary extract from which a number of compounds are isolated as a mixture. Subsequently, in a following second step, the mixture of compounds isolated from the first extract is further treated, for example extracted, to produce a second extract from which is isolated a mixture containing predominantly the desired compounds. Subsequently, in a following third step, the mixture of compounds isolated from the second extract is further treated, for example using chromatography to remove undesired compounds and to produce a mixture containing the desired compounds in a solution with a chromatography mobile phase. The desired compounds are then isolated from the mobile phase by, for example, a distillation method. However, such multiple step processes are time consuming and expensive. Furthermore, subjecting some extracts to further processes is undesirable since it may result in contamination, loss or damage of the desired materials.

Hemp and/or Industrial hemp is a variety of the Cannabis sativa plant species that is grown specifically for the industrial uses of its derived products. Industrial hemp is a strain of Cannabis sativa which includes a lower concentration of psychoactive tetrahydrocannabinol (THC) and a higher concentration of cannabidiol (CBD).

THC is a controlled drug which is stringently regulated worldwide. The maximum THC content (as the THC acid—ie THCA) levels permitted in industrial hemp are <0.2 wt % on a dry matter basis in Europe and USA and 0.3% wt % in Canada and 1% in Switzerland. The CBD:THC ratio is normally in the region of 10:1 to 20:1 and in rare cases, for example GMO material, it may be higher. However, for a plant to express more than about 3% CBD as the acid in the dry leaf and buds it will, inevitably, also produce higher THC content than the legally subscribed limits of 0.2% in Europe and USA or 0.3% in Canada. Thus, industrial hemp must inevitably include relatively low levels of CBD, to ensure the THC level does not exceed the permitted level. As a result, isolation of CBD from industrial hemp in economically viable yields and acceptable purities is very difficult to achieve and primary extracts produced by any of the traditional extraction methods require expensive and low yielding downstream purification steps.

Processes which may be used for extraction of compounds from Cannabis plants (but which are not believed to be used for treatment of industrial hemp) involve solvent extraction.

One solvent extraction process may involve use of ethanol or hexane. However, such solvents are found to extract a wide range of components from Cannabis plants including a range of cannabinoids together with phytowaxes, polyphenols, chlorophyll and other coloured substances. If such solvents were used to treat industrial hemp, a relatively low level of CBD and/or relatively low purity CBD would be extracted which would require extensive downstream purification. Furthermore, given the solvents are generally removed by vacuum evaporation at an elevated temperature, any fragile and/or volatile components (e.g. low molecular weight terpenoids) in the extract would be lost from the extract. Consequently, use of the process described is not economically viable for producing CBD-rich and/or Botanical Drug Substances.

An alternative extraction process may involve use of supercritical (sc) fluids, for example scCO₂. However, in view of the high operating pressure and elevated temperature required, use of such an extraction process is costly in terms of equipment and operating costs. In addition, use of scCO₂ is found to co-extract significant amounts of phytowaxes which necessitates a downstream, “winterization” step. Use of scCO₂ also may disadvantageously extract chlorophyll. Furthermore, it is found that the acidic nature of the scCO₂ tends to cause degradation of mono- and di-terpenes (which may be in Cannabis plants) which is undesirable since such terpenes may act synergistically (in a so-called “entourage effect”) with CBD in a dietary or health supplement or BDS which includes CBD and such terpenes. Thus, it is preferable to preserve such terpenes in any extracts of Cannabis plants, for example of industrial hemp.

It is an object of preferred embodiments of the present invention to address the above described problems.

It is an object of preferred embodiments of the present invention to provide an improved process for treatment of industrial hemp.

It is an object of preferred embodiments of the present invention to provide a process for extracting CBD and/or CBDA from a biomass at relatively high purity and relatively high yield.

It is an object of preferred embodiments of the invention to provide a simple and efficient process for preparing dietary ingredients, dietary or health supplements or BDSs from CBD and/or CBDA-containing plant material and/or from industrial hemp.

According to a first aspect of the invention, there is provided a method of extracting at least one cannabinoid from a biomass, the method comprising the following steps:

(i) contacting the biomass with a solvent formulation which comprises a C₁₋₄ fluorinated hydrocarbon or a C₁₋₄ hydrofluorocarbon ether, thereby to charge the solvent formulation with an extract from the biomass; and

(ii) separating charged solvent formulation from the biomass.

The method has been found to be particularly efficient at extracting high purity cannabinoids from the biomass which thereby may not require extensive downstream purification prior to use as dietary or health supplements or as a BDS.

Said cannabinoid may be naturally-occurring in the biomass or may be a derivative of a cannabinoid which is naturally-occurring in the biomass. When said cannabinoid is a derivative, the method may include actively treating the biomass prior to step (i) of the method to derivatise a naturally-occurring cannabinoid in the biomass, as described hereinafter.

Said biomass may comprise or preferably consist essentially of industrial hemp and/or industrial hemp which has been treated, for example to derivatise (e.g. to decarboxylate) one or more cannabinoids included in the hemp.

Unless otherwise stated, weights and wt % of cannabinoids and/or components in a biomass referred to herein are assessed on a dry matter basis.

As foreshadowed, cultivation and use of hemp is controlled by legislation to ensure its THC content does not exceed a predetermined upper level. Said biomass preferably complies with relevant legislation, for example EU Commission Delegated Regulation (EU) 2017/1155 of 15 Feb. 2017 and/or any legislation which replaces such legislation. Said biomass preferably complies with relevant legislation in other countries.

Said biomass preferably includes a THC content of less than 0.3% or preferably less than 0.2%, (except for Switzerland where the upper limit is 1.0%) suitably measured as described in Annex 3 of EU Regulations EU 2017/1155. Said biomass used in the method may comprise a said biomass which includes a THC content of less than 0.3% or preferably less than 0.2% as described or is derived from such a biomass. Although legislation and/or references sometimes refer to THC instead of THCA (e.g. the material naturally occurring in a biomass prior to decarboxylation), a skilled person in the art is aware of conventions in the art.

Said biomass preferably comprises hemp (or is derived from hemp, for example by decarboxylation) which complies with Article 9 of Delegated Regulation (EU) 2017/1155 or any succeeding legislation.

Said biomass may comprise leaves and/or flowers (preferably obtained from industrial hemp) which comprise one or more cannabinoids and/or derivatives of one or more cannabinoids (e.g. decarboxylated derivatives) produced by treatment, for example a decarboxylation treatment of said leaves and/or flowers. The sum of the wt % of leaves and flowers in said biomass may be at least 50 wt %, at least 75 wt %, at least 95 wt %, at least 98 wt %, or about 100 wt %.

Said biomass preferably includes CBD and/or CBDA. In said biomass, a sum (S1) of the wt % of CBD and CBDA on a dry matter basis may be at least 0.3 wt % and it may be less than 4 wt %, less than 3.6 wt % or less than 3.1 wt %. Said sum (S1) may be in the range 0.3 to 4 wt %, preferably in the range 0.5 to 3 wt %,

Said biomass may include THC and/or THCA. In said biomass, a sum (S2) of the wt % of THC and THCA on a dry matter basis may be less than 0.3 wt %, preferably less than 0.2 wt %. Said sum (S2) may be at least 0.001 wt % or at least 0.01 wt %.

The sum of sum (S1) and sum (S2) may be at least 0.3% wt and it may be less than 4 wt %, less than 3.6 wt % or less than 2.1 wt %. Said sum of sum (S1) and sum (S2) may be in the range 0.3 to 5 wt %, preferably in the range 0.5 to 4 wt %, more preferably in the range 1.0 to 3.0 wt %.

A ratio defined as the sum (S1) divided by the sum (S2) may be at least 3, preferably at least 6, more preferably at least 8 or at least 9. The ratio may be less than 50, less than 30, or less than 15. Preferably, the ratio is in the range 6 to 20, for example about 10.

In an embodiment (A), said biomass may not be actively treated prior to step (i) to derivatise a naturally-occurring cannabinoid in the biomass. For example, said biomass may not be actively treated to decarboxylate a naturally-occurring cannabinoid included in the biomass. A reference to “actively treated” or a cognate expression refers to a treatment which is operated or operable by a person using man-made equipment. It suitably excludes, for example, treatment and/or a reaction which occurs under ambient conditions, such as ambient temperature. Some cannabinoids may react, for example, decarboxylate to some extent under ambient conditions but, in the context, this is not regarded as an active treatment.

In embodiment (A), preferably, no component of said biomass is derivatised (e.g. oxidized or decarboxylated) in an active treatment prior to step (i) of the method.

In embodiment (A), the ratio of the wt % of CBDA divided by the wt % of CBD in the biomass is preferably greater than 1, preferably greater than 5, more preferably greater than 10. It may be less than 500.

In an embodiment (B), said biomass may be treated, for example actively treated, prior to step (i) to derivatise a naturally-occurring cannabinoid in the biomass. The biomass may be treated to decarboxylate one or more cannabinoid compounds included in the biomass. For example, the biomass may include cannabidiolic acid (CBDA) and may include tetrahydrocannabinolic acid (THCA) and/or other cannabinoid acids. Such compounds may be decarboxylated when within the biomass, to yield tetrahydrocannabinol (THC) and cannabidiol (CBD) respectively.

In embodiment (B), said biomass (suitably comminuted biomass) may be heated, for example to a temperature of greater than 80° C. or greater than 100° C., preferably (but not necessarily) in a substantially inert atmosphere (e.g. under a nitrogen blanket) for a period of time (e.g. in excess of 30 minutes or in excess of 1 hour) thereby to decarboxylate one or more major components in the biomass. After treatment of the biomass, it may be subjected to step (i) of the method as described.

In embodiment (B), the ratio of the wt % of CBD divided by the wt % of CBDA in the biomass is suitably greater than 1, is preferably greater than 5 and, more preferably, is greater than 10. It may be less than 500.

Said biomass, prior to any decarboxylation, for example in an active treatment as described, may include CBDA and/or THCA. Preferably, in said biomass prior to any decarboxylation, for example in an active treatment as described, the sum of the wt % of CBDA and THCA is at least 0.2 wt %. Said sum may be less than 5 wt % or less than 3.5 wt %.

After decarboxylation, the sum of the wt % of CBD and THC in the biomass may be at least 0.2 wt %. Said sum may be less than 5 wt % or less than 3.5 wt %.

After decarboxylation, said biomass may include at least 0.3 wt %, preferably at least 1 wt % of CBD. In said biomass after decarboxylation, CBD may be present at a level of less than 5 wt % or less than 4 wt %.

Said biomass treated in step (i) may have a water content of less than 10 wt % or less than 5 wt %.

In step (i), contact (e.g. initial contact) of biomass with solvent formulation may take place when the biomass is at ambient temperature or optionally at less than ambient temperature, for example less than 10° C., suitably less than 5° C., optionally less than 0° C., or less than −5° C. Thus, the biomass may be kept at ambient temperature, or cooled optionally to a temperature of less than ambient temperature prior to contact (e.g. initial contact) with said solvent formulation. Such cooling may be achieved by placing the biomass in a refrigerator or freezer. The temperature of said biomass on contact (e.g. initial contact) with said solvent formulation may be at least −20° C., preferably at least −14° C.

The solvent formulation which contacts (e.g. initially contacts) the biomass may be at ambient temperature or less than ambient temperature, for example less than 15° C., suitably less than 10° C., preferably less than 5° C., more preferably less than 0° C., especially less than −2° C. Said temperature of said solvent formulation may be greater than −20° C., suitably greater than −15° C., preferably greater than −12° C.

Said solvent formulation may be maintained at a temperature as described, especially a temperature of less than 5° C., less than 0° C. or less than −2° C., for a period of at least 10 minutes, preferably at least 30 minutes, more preferably at least 1 hour, especially for at least 1.5 hours. Said solvent formulation may be passed through the biomass multiple times. For example, it may be circulated and/or re-circulated through the biomass, suitably whilst maintaining the temperature of the solvent formulation at ambient temperature or a temperature of less than 5° C., preferably less than 0° C., more preferably less than −2° C. The low temperature described is found to be advantageous for extracting desired cannabinoids (especially CBD and/or CBDA) from the biomass, at high purity.

In the method, said biomass may be arranged in a receptacle between an inlet and outlet of the receptacle. In the method, solvent formulation may pass into the receptacle via said inlet, through the biomass and out of the receptacle via said outlet.

Said biomass arranged in said receptacle may be in any suitable form. The form is suitably selected to optimise extraction of components therefrom. Thus, biomass may be processed to adjust its form for use in the process. For example, solid material may be comminuted and such comminuted material may be arranged in the receptacle. In preferred embodiments, the mass of material arranged in the receptacle is in a finely divided form.

The method of the first aspect may involve selecting a biomass which includes components to be extracted and packing the biomass into free space in said receptacle so that said biomass extends over a length of at least 5 cm, preferably at least 20 cm, more preferably at least 40 cm in said receptacle, preferably in a column. Said biomass preferably extends to an upper region of the receptacle and a lower region thereof and, suitably, only the biomass is present in the receptacle between said upper and lower regions. Only after said mass of material has been packed into said receptacle so that it extends over said length described is a solvent formulation passed into said receptacle.

Said receptacle may include an internal diameter (ID) (which is preferably substantially constant) in which said biomass is arranged and a length (L) over which the biomass extends wherein, preferably, the aspect ratio of biomass in the receptacle defined as L/ID is at least 10, at least 20 or at least 25. Said aspect ratio may be less than 200, or less than 120.

Said biomass may be packed into said receptacle at a density of at least 0.25 g/cm³, preferably at least 0.30 g/cm³, more preferably at least 0.35 g/cm³, especially at least 0.40 g/cm³. The density of the biomass may be achieved by use of a ram (or the like) to compress the biomass. The biomass is suitably substantially immovable when in position. The biomass is suitably substantially static during the flow of said solvent formulation therethrough.

Said solvent formulation may be passed through the biomass at a rate of at least 0.02 ml/minute per gram of said biomass in the receptacle. Said rate may be less than 1 ml/minute per gram. The flow rate may be at least 0.5 BV/hour where “By” refers to the bed volume. Thus, the unit refers to the volume of said solvent formulation passing through the biomass per hour divided by the volume taken up by the biomass. The flow rate is preferably 10 BV/hour or less.

As described, said solvent formulation comprises a C₁₋₄ fluorinated hydrocarbon or a C₁₋₄ hydrofluorocarbon ether.

A said hydrofluorocarbon ether preferably comprises one or more carbon, fluorine, hydrogen and oxygen atoms only. It may include up to 10, preferably up to 8, more preferably, up to 6, fluorine atoms. It preferably includes at least 2, more preferably at least 3 fluorine atoms. It is preferably aliphatic and/or saturated. An example of a hydrofluorocarbon ether is 1,1,1,2,2-pentafluorethyl methyl ether.

Said C₁₋₄ fluorinated hydrocarbon is preferably non-chlorinated. Preferably, it comprises one or more carbon atoms, one or more fluorine atoms together with one or more other atoms selected from hydrogen atoms and iodine atoms. More preferably, it comprises one or more carbon, fluorine and hydrogen atoms only. Preferably, said fluorinated hydrocarbon is a C₁₋₃, more preferably a C₂₋₃, fluorinated hydrocarbon. Especially preferred is a C₂ fluorinated hydrocarbon.

Said fluorinated hydrocarbon may include 10 or fewer, suitably 8 or fewer, preferably 7 or fewer, more preferably 5 or fewer, especially 4 or fewer, fluorine atoms. Preferably, said fluorinated hydrocarbon includes at least 2, more preferably at least 3, fluorine atoms.

Said fluorinated hydrocarbon is preferably aliphatic. It is preferably saturated.

Said fluorinated hydrocarbon may have a boiling point at atmospheric pressure of less than 20° C., preferably less than 10° C., more preferably less than 0° C., especially less than −10° C. The boiling point may be greater than −90° C., preferably greater than −70° C., more preferably greater than −50° C., especially greater than −40° C.

Said solvent formulation may comprise a solvent selected from: iodotrifluoromethane, CF₃H (HFC-23, trifluoromethane), CH₃F (HFC-41, fluoromethane), CH₂F₂ (HFC-32, difluoromethane), CF₃CF₂H (HFC-125, pentafluoroethane), CF₃CH₃(HFC-143 A, 1,1,1-trifluoroethane), HCF₂CH₃(HFC-152 A, 1,1-difluoroethane), CF₃CHFCF₃(HFC-227 EA, 1,1,1,2,3,3,3-heptafluoropropane), CF₃CF₂CF₂H (HFC-227 CA, 1,1,1,2,2,3,3-heptafluoropropane), CF₃CH₂CF₃(HFC-236 FA, 1,1,1,3,3,3-hexafluoropropane), CF₃CF₂CH₃(HFC-245 CB, 1,1,1,2,2-pentafluoropropane), CF₃CF₂CH₂F (HFC-236 CB, 1,1,1,2,2,3-hexafluoropropane), HCF₂CF₂CF₂H (HFC-236 CA, 1,1,2,2,3,3-hexafluoropropane), CF₃CHFCF₂H (HFC-236 EA, 1,1,1,2,3,3-hexafluoropropane), and CH₂FCF₃(HFC-134A, 1,1,1,2-tetrafluoroethane).

Preferably, said solvent formulation comprises a solvent selected from: iodotrifluoromethane, 1,1,1,2,3,3,3-heptafluoropropane (HFC-227 EA), 1,1,1,2,2,3,3-heptafluoropropane (HFC-227CA) and 1,1,1,2-tetrafluoroethane (HFC-134a).

More preferably, said solvent formulation comprises a solvent selected from: 1,1,1,2,3,3,3-heptafluoropropane (R-227EA) and 1,1,1,2-tetrafluoroethane, with 1,1,1,2-tetrafluoroethane being especially preferred.

Said C₁₋₄ fluorinated hydrocarbon or C₁₋₄ hydrofluorocarbon ether preferably has a purity of at least 98% w/w.

Said solvent formulation is preferably in a liquid state when contacted with said biomass in step (i) of the method. Said solvent formulation is preferably in a sub-critical state when contacted with said biomass in the method.

Said solvent formulation preferably includes a major amount of C₁₋₄ fluorinated hydrocarbon or C₁₋₄ hydrofluorocarbon ether. Said solvent formulation suitably includes at least 70 wt %, preferably at least 80 wt %, more preferably at least 90 wt %, especially at least 92 wt % of a said C₁₋₄ fluorinated hydrocarbon or C₁₋₄ hydrofluorocarbon ether. Said solvent formulation preferably includes a single type of C₁₋₄ fluorinated hydrocarbon or C₁₋₄ hydrofluorocarbon ether. Said solvent formulation preferably includes a single type of C₁₋₄ fluorinated hydrocarbon and no C₁₋₄ hydrofluorocarbon ether. Said solvent formulation preferably includes a major amount (e.g. at least 70 wt %, preferably at least 80 wt %, more preferably at least 90 wt %, especially at least 92 wt % of a C₁₋₄ fluorinated hydrocarbon, especially HFC 134a.

In some embodiments, said solvent formulation may include at least 95 wt %, preferably at least 97 wt %, more preferably at least 99 wt % of a C₁₋₄ fluorinated hydrocarbon, especially HFC 134a. In such embodiments, said solvent formulation may consist essentially of a single C₁₋₄ fluorinated hydrocarbon, especially HFC 134a.

Where the solvent formulation does not consist essentially of a single solvent, the solvent formulation may include a modifier to adjust the properties of the solvent formulation.

Said modifier may comprise any material which is capable of modifying the properties of the solvent formulation thereby to affect extracts obtained from the biomass. Said selected modifier may affect the pH of the solvent formulation. Preferably, said selected modifier comprises a co-solvent. A said co-solvent may be an additional C₁₋₄ fluorinated hydrocarbon or C₁₋₄ hydrofluorocarbon ether. Preferably, said solvent formulation includes a modifier selected from: a C₂₋₆ hydrocarbon such as an alkane or cycloalkane with alkanes such as ethane, n-propane, i-propane, n-butane and i-butane being especially preferred; and hydrocarbon ethers, particularly dialkylethers such as dimethylether, methylethylether and diethyl ether. In other embodiments, said modifier may be polar, for example having a dielectric constant, at 20° C., of greater than 5. Such modifier may be selected from: amides, especially N,N′-dialkylamides and alkylamides, with dimethylformamide and formamide being preferred; sulphoxides, especially dialkyl sulphoxides, with dimethylsulphoxide being preferred; alcohols, especially aliphatic alcohols for example alkanols, with methanol, ethanol, 1-propanol and 2-propanol being preferred; ketones, especially aliphatic ketones, for example dialkyl ketones, with acetone being especially preferred; organic acids, especially carboxylic acids with formic acid and acetic acid being preferred; carboxylic acid derivatives, for example anhydrides, with acetic anhydride being preferred; cyanide derivatives, for example hydrogen cyanide and alkyl cyanides, with methyl cyanide and liquefied anhydrous hydrogen cyanide being preferred; ammonia; sulphur containing molecules including sulphur dioxide, hydrogen sulphide and carbon disulphide; inorganic acids for example hydrogen halides with liquefied anhydrous hydrogen fluoride, chloride, bromide and iodide being preferred; nitro derivatives, for example nitroalkanes and nitroaryl compounds, with nitromethane and nitrobenzene being especially preferred.

A preferred modifier may have a boiling point of at least −30° C., for example −26° C.; and preferably less than 10° C., or less than 2° C. A preferred modifier may have a melting point of greater than −160° C., for example greater than −150° C.; and preferably less than −100° C. or less than −120° C. A preferred modifier includes carbon and hydrogen atoms and, optionally, an oxygen atom. A preferred modifier is saturated. A preferred modifier is selected from an alkane and an ether. A preferred modifier has a molecular weight of at least 30, preferably at least 40, more preferably at least 44; and a molecular weight of less than 100, preferably less than 80, more preferably less than 65.

Said solvent formulation may include up to 20 wt %, preferably up to 10 wt %, more preferably up to 8 wt % of modifier (preferably a single type of modifier). In some embodiments, said solvent formulation may include no modifier.

Said solvent formulation may include only a single type of modifier. Said solvent formulation may consists essentially of a single solvent selected from a C₁₋₄ fluorinated hydrocarbon (said solvent preferably being HFC 134a) and C₁₋₄ hydrofluorocarbon ether and a single modifier (said modifier preferably being an alkane or ether, with butane and dimethylether being especially preferred).

In a preferred embodiment, said solvent formulation consists essentially of a single solvent (eg said single solvent makes up more than 99 wt % or more than 99.5 wt % or more than 99.9 wt % of said solvent formulation) which is selected from a C₁₋₄ fluorinated hydrocarbon (said solvent preferably being HFC 134a) and C₁₋₄ hydrofluorocarbon ether. Thus, preferably said solvent formulation does not include any type of modifier. In a preferred embodiment, said method includes isocratic treatment of said biomass, preferably with a C₁₋₄ fluorinated hydrocarbon (especially HFC134a).

The conditions referred to above under which said biomass is contacted with said solvent formulation are referred to as a or said “first set of conditions”.

After contact under said first set of conditions, said biomass may be contacted with a solvent formulation under a second set of conditions.

Said second set of conditions may differ from said first set of conditions in at least one variable selected from:

(A) the physical state of the solvent formulation; and

(B) a chemical property of the solvent formulation.

As described in (A) above, the physical state of the solvent formulation may be varied during extraction of said biomass, for example in the receptacle. described the temperature or pressure of said solvent formulation may be varied. If the physical state of the solvent formulation is to be varied, it is preferred that the temperature of the solvent formulation is varied. For example, said first set of conditions may involve the solvent formulation being at a relatively low first temperature, as described. The temperature may be raised so that said solvent formulation is at a second temperature, greater than the first temperature. The temperature may be further raised so that said second set of conditions involve the solvent formulation being at a third temperature greater than the second temperature. Each of the temperatures of the solvent formulation may be within the range −10° C. to 60° C.

As described in (B) above, a chemical property of the solvent formulation may be varied during extraction of said mass of material in the receptacle. This may be achieved by including varying amounts of a selected modifier, as described herein, in said solvent formulation. It is though preferred not to use a modifier, but to carry out the extraction of the biomass using a single type of solvent as described.

Preferably, in the method, when a second set of conditions is used, the physical state of the solvent formulation, especially the temperature thereof, is varied as described in (A) above.

A receptacle in which said biomass is preferably arranged in the method is preferably a column. The column preferably has a circular cross-section between its inlet and outlet. The column preferably has a substantially constant cross-sectional area and shape between its inlet and outlet. The column may have an inside diameter of at least 2 cm, preferably at least 4 cm, more preferably at least 5 cm. The diameter of the column may be less than 30 cm, preferably less than 15 cm. The length of the column between its inlet and outlet may be at least 40 cm, preferably at least 50 cm, more preferably at least 75 cm, especially at least 100 cm. The length of the column may be less than 500 cm, preferably less than 400 cm, more preferably less than 250 cm. The column may have a length:inside diameter ratio of greater than 1:1; and preferably less than 100:1. The length:diameter ratio may be at least 10, preferably at least 20. The column preferably has a circular cross-section over at least 50%, preferably 80% of its length. It preferably has a circular cross-section over substantially the entirety of its length. The length:inside diameter ratio may be less than 50:1. The column is preferably made out of metal, such as steel.

The material extracted from the biomass and charged to the solvent is preferably a compound which occurs naturally in the biomass or is a derivative (e.g. a decarboxylated derivative) of a compound which occurs naturally in the biomass.

In the method, after step (ii), the charged solvent formulation may be treated so solvent formulation is separated from the extract, suitably to isolate the extract. Separation may simply involve evaporation of the solvent formulation.

The method may include preparing separate extracts from said biomass, for example, using said first set of conditions and a second set of conditions as described. The separate extracts may be isolated separately (thereby to provide extracts which may have different compositions, for example different amounts of CBD and/or CBDA or the separate extracts may be combined to define a single extract.

In some extraction processes (e.g. involving treatment of biomasses containing cannabinoids), an extract may be treated, for example, with a view to increasing the amount of one or more desired cannabinoids in a product of the treatment. Such treatment may comprise dissolution of an extract in a solvent (e.g. an organic solvent, such as ethanol) and/or subjecting the extract to a temperature less than ambient temperature (e.g. less than 0° C., less than −10° C. or less than −15° C.), for a period of time, for example at least 1 hour, at least 10 hours or at least 20 hours. The intention of the treatment is to cause precipitation of high molecular weight components (e.g. waxes) from the extract, thereby to increase the concentration of desired cannabinoids in the extract. After the treatment, the extract may be treated, for example filtered (e.g. to capture the high molecular weight components) and the filtrate collected thereby to define a purified extract, relatively rich in desired cannabinoids. Whilst it is conventional to include such a treatment as described (which is often referred to as “winterization”), it is unexpectedly found, in accordance with preferred embodiments of the invention, that the method of the first aspect may be sufficiently selective so that significant quantities of high molecular weight components such as waxes, are not extracted in the method. Advantageously, a winterization process may not be required. Thus, in a preferred embodiment, the method preferably does not include a winterization process. In the preferred embodiment (referred to as “embodiment (C)”, the method preferably does not include one or more of the following:

(a) said extract (e.g. containing at least one cannabinoid, suitably CBD and/or CBDA) being treated, for example, with a view to increasing the amount of one or more desired cannabinoids in a product of the treatment;

(b) dissolution of an extract in a solvent (e.g. an organic solvent, such as ethanol);

(c) subjecting the extract to a temperature less than ambient temperature (e.g. less than 0° C., less than −10° C. or less than −15° C.), for a period of time, for example at least 1 hour, at least 10 hours or at least 20 hours;

(d) any other treatment intended to cause precipitation of high molecular weight components (e.g. waxes) from the extract;

(e) filtration (e.g. to capture the high molecular weight components).

Embodiment (C) preferably does not include at least two, at least three, at least four or any of steps (a) to (f) described. Embodiment (C) preferably does not include any treatment intended to cause or which does cause precipitation of high molecular weight components (e.g. waxes) from the extract.

Thus, in embodiment (C), the total weight of waxes in the charged solvent and/or the extract (e.g. containing CBA and/or CBDA) derived therefrom is less than the total weight of waxes in the biomass after the biomass has been treated in the method. Waxes may be any lipophilic, organic compounds with a melting point of greater than 40° C. They are suitably substantially insoluble in water. The wax ratio, defined as the total weight of waxes in the biomass after treatment in the method divided by the total weight of waxes in the charged solvent, for example after step (ii) (and for the avoidance of doubt without any low temperature, for example winterization, treatment described, is suitably at least 5, preferably at least 10, more preferably at least 20, especially at least 40. Thus, the extract may include a very small amount of wax at most and as a result does not need to be subjected to a winterization treatment.

The extract may include a low level of waxes at most—e.g. less than 0.5 wt %, less than 0.25 wt %, less than 0.1 wt % or less than 0.05 wt %.

In the extract, the cannabinoid ratio, defined as the total weight of non-wax based cannabinoids divided by the total weight of waxes in the extract may be at least 5, preferably at least 10, more preferably at least 50, especially at least 100.

The extract is preferably a mobile oil at 25° C.

When embodiment (A) is followed in the method, in preference to embodiment (B), the method may include an embodiment (D), wherein the extract is treated to derivatise a cannabinoid (e.g. CBDA) which was naturally-occurring in the biomass and is present in the extract. In this case, the extract may be treated to derivatise such a naturally-occurring cannabinoid in the extract. The extract may be treated to decarboxylate one or more cannabinoid compounds included in the extract. For example, the extract may include cannabidiolic acid (CBDA) which may be decarboxylated when within the extract, to yield cannabidiol (CBD). In such treatment, said extract may be heated, for example to a temperature of greater than 80° C. or greater than 100° C., preferably in a substantially inert atmosphere (e.g. under a nitrogen blanket) fora period of time (e.g. in excess of 30 minutes or in excess of 1 hour) thereby to decarboxylate the CBDA.

In a second aspect, the invention extends to an extract from a biomass as described per se. The extract may include a low level of waxes at most—e.g. less than 0.5 wt %, less than 0.25 wt %, less than 0.1 wt % or less than 0.05 wt % of waxes. In the extract, the cannabinoid ratio, defined as the total weight of non-wax based cannabinoids divided by the total weight of waxes in the extract may be at least 5, preferably at least 10, more preferably at least 50, especially at least 100. The extract is preferably a mobile oil at 25° C.

In said extract, a ratio (A) defined as (the sum of the weights of CBD and CBDA in the extract):(the weight of terpenes in the extract) may be in the range 2:1 to 50:1. Ratio (A) may be in the range 2:1 to 40:1, for example 5:1 to 25:1. Advantageously, the process used to prepare the extract is found to readily extract most if not all the terpenes very quickly (ie 90 wt %-100 wt % of the terpenes present).

In said extract, a ratio (B) defined as (the sum of the weights of CBD and CBDA in the extract):(the weight of terpenes in the extract) may be in the range 2:1 to 50:1. Ratio (B) may be in the range 2:1 to 401, for example 5:1 to 25:1.

In said extract, a ratio (C) defined as (the sum of the weights of CBD and CBDA in the extract):(the sum of the weights of beta-caryophyllene, humulene, alpha-bisabolol, alpha-pinene, myrcene and limonene in the extract) may be in the range 2:1 to 50:1. Ratio (C) may be in the range 2:1 to 40:1, for example 5:1 to 25:1.

Although the majority of said solvent formulation used in the method of the first aspect is separated from the extract, it is possible some of said solvent formulation may remain, for example as a contaminant, in the extract. Thus, said extract may include at least 0.0001 wt %, or at least 0.0010 wt %. of said solvent formulation, for example comprising a said a C₁₋₄ fluorinated hydrocarbon or a C₁₋₄ hydrofluorocarbon ether, Said extract may include less than 0.1000 wt %%, or less than 0.01 wt % %, of a said solvent formulation.

Said extract may include at least 0.0001 wt % or at least 0.0010 wt %, of HFC134a. Said extract may include less than 0.1000 wt % or less than 0.01 wt %, of HFC134a.

Said extract is preferably a “mobile oil” at 25° C. or a mixture of viscous oil and solid or crystalline material

In a third aspect, the invention extends to a formulation which comprises a product of the method of the first aspect and/or an extract of the second aspect.

The formulation may be for the following:
As a health supplement;
As a dietary ingredient;
As a dietary supplement;
As a medically prescribed botanical drug substance;
As an OTC health related supplement. This may be for use as: a pain killer, a sleep aid, an antidepressant and an anti-anxiety remedy, anorexia treatment, a muscle relaxant, a spasticity aid, an anti-emetic, an appetite enhancer, an aid in some neurological disorders such as involuntary movements and vocalizations, a suppressant for involuntary tics and other Tourette symptoms;
Food additive, e.g. flavour enhancer;
Cosmetic and aroma ingredient;
Recreational uses.

Said formulation may include 1% to 5% vol of said extract.

Said formulation may be in the form of a liquid or solid. When in liquid form, the liquid may be in the form of an oral or nasal spray or a beverage. When in solid form, the solid may be in the form of a capsule, tablet or food product (e.g. confectionery product).

In a fourth aspect, there is provided a method of making a formulation, for example according to the third aspect, the method comprising:

(a) selecting a product of the method of the first aspect and/or an extract of the second aspect;

(b) contacting material selected in step (a) with one or more other components of the formulation so as to incorporate a predetermined concentration of cannabinoids (especially CBD) in the formulation;

(c) producing a mixture of said selected material and one or more other components.

The invention extends to the use of an extract of the second aspect, formulation of the third aspect or product of the method of the fourth aspect as a dietary ingredient, dietary supplement, health supplement or BDS.

Any feature or any aspect of any invention or embodiment described herein may be combined with any feature of any aspect of any other invention or embodiment described herein mutatis mutandis.

Specific embodiments of the invention will now be described, by way of example, with reference to the accompanying diagrammatic drawings, in which:

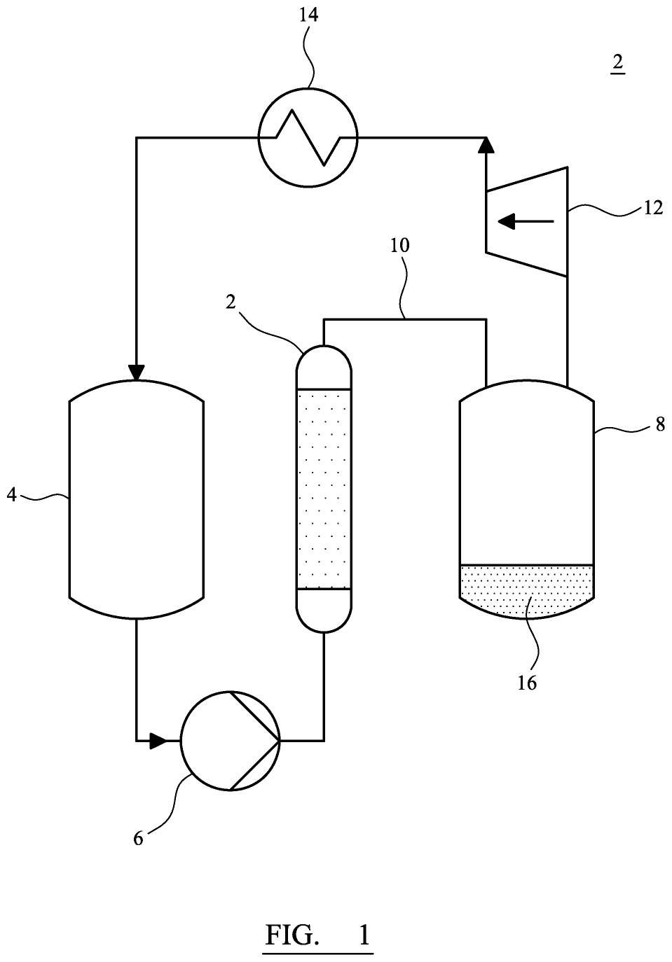

FIG. 1 is a schematic representation of apparatus for carrying out extraction of a cannabinoid-containing biomass; and

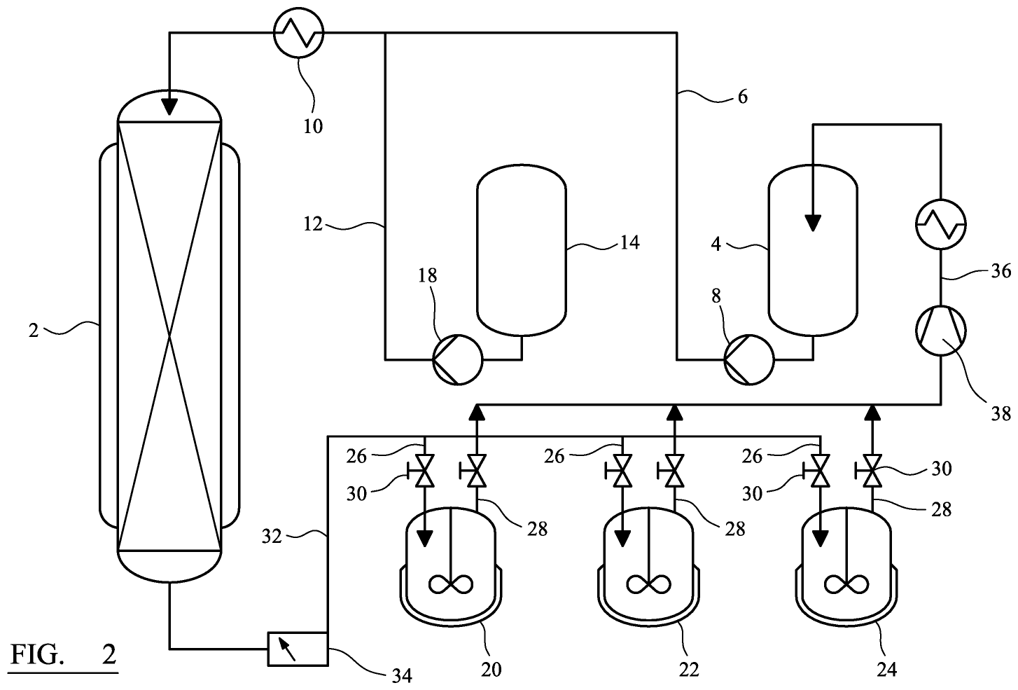

FIG. 2 is an alternative apparatus for carrying out extraction of a cannabinoid-containing biomass.

The following materials are referred to hereinafter:

R134a—refers to 1,1,1,2-tetrafluoroethane.

Hemp-type L 2.1—a commercially available industrial hemp.

Hemp-type L 1.7—a commercially available industrial hemp.

The following abbreviations used herein have the meanings stated.

| Abbreviation | Meaning |
|---|---|
| THC | tetrahydrocannabinol |
| THCA | tetrahydrocannabinolic acid |
| CBD | cannabidiol |
| CBDA | cannabidiolic acid |
| CBG | cannabigerol |
| CBGA | cannabigerolic acid |
| CBN | cannabinol |
| CBDV | cannabidivarin |
| THCV | tetrahydrocannabivarin |
| CBC | cannabichromene |

Hempflax A hemp—a commercially available industrial hemp, with the following composition:

| Composition | Amount (wt %) |
|---|---|
| THC | <0.05 |
| CBD | 0.148 |
| THCA | <0.05 |
| CBDA | 0.392 |
| CBGA | <0.05 |
| CBG | <0.05 |
| CBN | <0.05 |

Referring to FIG. 1, apparatus 2 for carrying out extraction of a biomass consisting of industrial hemp containing bio-cannabinoids comprises a jacketed stainless steel extraction column 2 having an internal diameter of 3.5 cm and a height of 1 m. Upstream of column 2 is a solvent storage vessel 4 with a liquid metering pump 6 being arranged between vessel 4 and column 2 for circulating liquid within the apparatus in which biomass to be extracted is tightly packed.

Downstream of column 2 is a collection/evaporation vessel 8 which communicates with the top of column 2 via pipe 10.

Downstream of vessel 8 is an oil free gas compressor 12 and a monitoring device (not shown) to monitor and analyse fluid flowing downstream of vessel 8. Downstream of the compressor and monitoring device 12 is an in-line heat-exchanger 14 which is arranged to re-liquefy fluid prior to return to vessel 4.

The apparatus described was used in examples which follow.

### Example 1—General Method for Decarboxylating Cannabinoid-Containing Biomass

Semi-dried leaves and flowering parts of industrial hemp biomass, having water content of ca. 10% or less were selected. A belt dryer (Alco Food type AGT-400/900-E) was used to decarboxylate the cannabinoids present, at a temperature of 110-140° C., biomass bed thickness of between 3-10 mm, a throughput of 500 g/Hr and a treatment time of 1.0-1.5 Hrs.

Alternative apparatus that may be used for carrying out this step include ribbon blenders, paddle mixers, tumbler type mixers and screw type heat exchangers

### Examples 2 and 3

Following the general method of Example 1, Hemp-types L 2.1 (Example 2) and L 1.7 (Example 3) were decarboxylated and results are provided in the tables below.

### Results for Example 2:

| | Biomass Assay | Biomass Assay | Con- |
|---|---|---|---|
| | Pre-decar- | Post decar- | version |
| | boxylation | boxylation | (%) |
| Total cannabinoids | 1.98 wt % | 1.95 wt % | 98.4 |
| Total CBD(A) | 1.93 wt % | 1.82 wt % | 94.3 |
| | CBDA | CBD | |
| Total THC(A) | 0.001 wt % | 0.027 wt % | |
| | THCA | THC | |

### Results for Example 3:

| | Biomass Assay | Biomass Assay | Con- |
|---|---|---|---|
| | Pre-decar- | Post-decar- | version |
| | boxylation | boxylation | (%) |
| Total cannabinoids | 1.59 wt % | 1.38 wt % | 99 |
| Total CBD(A) | 1.55 wt % | 1.31 wt % | 99 |
| | CBDA | CBD | |
| Total THC(A) | 0.001 wt % | 0.027 wt % | |
| | THCA | THC | |

It will be appreciated for Examples 2 and 3 that, in both cases, the amount of THC in the biomass is very low.

The following examples describe methods and results for extracting a range of samples of industrial hemp under a range of conditions.

### Example 4

A sample of dried (typically containing 1-8 wt % water) industrial fibre hemp (250.2 g) (Hempflax A hemp) with a CBDA content of 0.5 wt % and THCA content of <0.1 wt % was decarboxylated as described in Example 1, milled to powder particle size of 1-2 mm and packed tightly into a stainless steel extraction column having dimensions of 3.5 cm internal diameter and 1 m length and the column was sealed. It was then cooled to −10° C. by placing in a freezer. The equipment was then reassembled, evacuated and storage vessel 4 was charged with HFC134a (approx. 4-6 Kg). Chilled ethylene glycol was circulated through the jackets of the extraction column and the storage vessel, until the temperature of the HFC 134a in the storage vessel reached −5° C. The chilled HFC134a was then percolated through the biomass in column 2 at a flow rate of 3 Kg/hour for fraction 1 extract and for fraction 2 extract, with the flow being directed out of the column 2 and into vessel 8. The HFC134a was continuously evaporated from the vessel 8 using a gas compressor 12 in FIG. 1 and recycled through a condenser 14 in FIG. 1 back into the vessel 4. Extracts were collected in two fractions: part 1 after 1 hour at 0° C. and HFC134a flow rate of 3 Kg/Hr and part 2 collected after a further 6 hours at 25° C. at the same flow rate. In each case, the product was harvested from the evaporation vessel by dissolving in a minimum volume of ethanol. (Note that ethanol was used for harvesting since the vessel in which the product was collected was too large for the small amount of product. There is no need to use ethanol in harvesting when product is produced on a larger and/or industrial scale in an appropriately-sized vessel). Both ethanolic fractions were analysed and results are reported below.

It was found that the CBDA content pre decarboxylation was 1.4 g; and the CBD content post decarboxylation was 1.14 g (appreciating that CBD has a lower molecular weight than CBDA).
Results of analyses are provided in the table below.

| | | CBD | Purity as | |
|---|---|---|---|---|
| | Weight | Content | CBD | Recovery |
| Sample | (g) | (g) | (wt %) | (%) |
| Feed Biomass | 250.2 | 1.14 | 0.5 | — |
| Spent Biomass | 249.6 | 0.2 (i.e. CBD | 0.08 | 12 |
| | | remaining in | | |
| | | spent biomass) | | |
| Fraction 1 (F1), | 2.0 | 0.6 | 30 | 52.6 |
| extracted at 0° C. | | | | |
| Fraction 2 (F2), | 1.9 | 0.3 | 18.1 | 26.3 |
| extracted at 25° C. | | | | |
| Fraction F1 + | 3.9 | 0.9 | | 79.9 |
| Fraction F2 | | | | |

Total yield=79.9%
Total mass balance=91.9%

It is noted that, in the experiments, the ethanol referred to is used as a diluent for HPLC analysis used and is accounted for in the calculations.

Thus, it should be appreciated from the example that the process can be used to produce a high CBD yield since, in the combined extracts, the total yield of CBD was 79.9 wt %. The product produced was a honey-coloured, mobile viscous oil with a rich aroma typical of a presence of terpenes.

### Example 5

The procedure described in Example 4 was generally followed on an industrial hemp sample which had been decarboxylated as described in Example 1. Six fractions were collected at 11-minute intervals using the same flow rate and temperature.

Results are provided in the table below which includes analysis of cannabidiol (CBD), tetrahydrocannabinol (THC) and cannabigerol (CBG) contents.

| | | CBD | | | CBD | Cumulative | Cumulative |
|---|---|---|---|---|---|---|---|
| | Weight | Content | CBG | THC | Weight | CBD Weight | Recovery |
| Sample ID | (g) | (% wt) | (% wt) | (% wt) | (g) | (g) | (%) |
| Biomass | 290 | 0.356 | <0.1 | <0.1 | 1.03 | | |
| Spent biomass | 287 | 0.12 | — | — | 0.35 | | 33.9 |
| Fraction F1 | 0.38 | 38.7 | — | 0.60 | 0.15 | 0.15 | |
| Fraction F2 | 0.28 | 35.0 | — | 0.80 | 0.10 | 0.25 | |
| Fraction F3 | 0.48 | 26.5 | 1.75 | 0.74 | 0.13 | 0.38 | |
| Fraction F4 | 0.38 | 21.8 | 1.84 | 0.53 | 0.08 | 0.46 | |
| Fraction F5 | 0.48 | 16.5 | 2.10 | 0.35 | 0.08 | 0.54 | |
| Fraction F6 | 0.50 | 13.0 | 2.05 | 0.21 | 0.07 | 0.61 | 59.2 |

CBD Extraction yield=59.2 wt %
Average THC content=0.47 wt %
Mass Balance=93.1 wt % (which is a good result considering physical losses are inevitable at the scale used).

It should be noted that the extracts are relatively concentrated in CBD and include a relatively low amount of THC. Typically, in use, the target CBD concentration might be around 2 wt %, resulting in a very low concentration of associated THC.

### Example 6

The procedure described in Example 5 was followed on another industrial hemp sample.
Results are provided in the table below.

| | | | | Cumulative | |
|---|---|---|---|---|---|
| | | CBD | CBD | CBD | Cumulative |
| | Weight | assay | Weight | Weight | Recovery |
| Sample ID | (g) | (% wt) | (g) | (g) | (%) |
| Biomass | 302 | 0.339 | 1.025 | | |
| Spent | 300 | Not | | | |
| biomass | | available | | | |
| Fraction F1 | 1.10 | 46.90 | 0.51 | 0.51 | 49.8 |
| Fraction F2 | 0.50 | 37.50 | 0.19 | 0.70 | 68.3 |
| Fraction F3 | 0.34 | 23.40 | 0.08 | 0.78 | 76.1 |
| Fraction F4 | 0.20 | 19.90 | 0.04 | 0.82 | 80.0 |
| Fraction F5 | 0.14 | 17.40 | 0.02 | 0.824 | 80.4 |

Total CBD extraction yield=80.4 wt %
Average THC content=0.25 wt %

Again, the process is found to produce a high CBD yield. The product was a honey-coloured, mobile oil, having an aroma typically of a terpene content.

### Example 7

The procedure generally described in Example 5 was followed using Hemp-type L 1.7. Results are provided in the tables below.

| | Weight | CBDA | CBG | CBD | THC | CBC |
|---|---|---|---|---|---|---|
| Sample ID | (g) | (% wt) | (% wt) | (% wt) | (% wt) | (% wt) |
| Biomass | 561.0 | 0.017 | 0.002 | 1.272 | 0.023 | 0.066 |
| Spent biomass | 539.0 | | | 0.14 | | |
| Fraction F1 | 7.50 | | 0.15 | 46.5 | 0.1 | 1.7 |
| Fraction F2 | 5.60 | | 0.18 | 31.0 | 1.1 | 2.16 |
| Fraction F3 | 3.90 | 0.02 | 0.25 | 18.0 | 0.84 | 2.0 |
| Fraction F4 | 6.00 | 0.02 | 0.22 | 15.0 | 0.56 | 1.04 |

| | | CBD | Cumulative | Cumulative |
|---|---|---|---|---|
| | Assay | Weight | CBD | recovery |
| | (% wt) | (g) | Weight (g) | (%) |
| Fraction F1 | 46.5 | 3.450 | 3.45 | 48.3 |
| Fraction F2 | 31.0 | 1.73 | 5.18 | 72.5 |
| Fraction F3 | 18.0 | 0.70 | 5.88 | 82.3 |
| Fraction F4 | 15.0 | 0.90 | 6.78 | 95.0 |

CBD Extraction yield=95.0 wt %. Note, this is a particularly high yield.

### Example 8

The procedure of Example 5 was followed using Hemp-type L 2.1. Results are provided in the tables below.

| | | CBD | CBD | Cumulative | Cumulative |
|---|---|---|---|---|---|
| | Weight | Assay | Weight | CBD | Recovery |
| Sample | (g) | (% wt) | (g) | Weight (g) | (%) |
| Biomass | 476.0 | 1.90 | 9.05 | | |
| Spent biomass | 464.0 | 0.42 | 1.95 | | 21.6 |
| Fraction F1 | 7.20 | 53.5 | 3.85 | 3.85 | 42.6 |
| Fraction F2 | 2.60 | 44.1 | 1.15 | 5.0 | 55.2 |
| Fraction F3 | 1.30 | 37.7 | 0.50 | 5.5 | 60.8 |
| Fraction F4 | 1.20 | 32.1 | 0.40 | 5.9 | 65.2 |
| Fraction F5 | 0.80 | 28.5 | 0.23 | 6.1 | 67.4 |

| | | | | | | | Total |
|---|---|---|---|---|---|---|---|
| | CBD | CBDa | THC | THCa | CBC | CBG | cannabinoids |
| Recovery | 66% | 1% | 89% | n/a | 73% | 57% | 65% |
| Mass | 87% | 111% | 110% | n/a | 96% | 74% | 88% |
| balance | | | | | | | |

### Example 9

The procedure of Example 5 was followed using Hemp-type L 1.7. Results are provided in the tables below.

| | | | | Cumulative | |
|---|---|---|---|---|---|
| | | CBD | CBD | CBD | Cumulative |
| | Weight | Assay | Weight | Weight | Recovery |
| Sample | (g) | (% wt) | (g) | (g) | (%) |
| Biomass | 505.0 | 1.22 | 6.14 | | |
| Spent | 480.0 | Not | | | |
| biomass | | available | | | |
| Fraction F1 | 7.90 | 45.39 | 3.59 | 3.59 | 58.4% |
| Fraction F2 | 4.00 | 25.87 | 1.04 | 4.63 | 75.4% |
| Fraction F3 | 2.80 | 15.06 | 0.42 | 5.05 | 82.2% |
| Fraction F4 | 3.70 | 8.46 | 0.3 | 5.35 | 86.3% |
| Fraction F5 | 3.00 | 6.10 | 0.2 | 5.55 | 90.4% |

| | CBDA | CBG | CBD | THC | CBC |
|---|---|---|---|---|---|
| Sample ID | (% wt) | (% wt) | (% wt) | (% wt) | (% wt) |
| Biomass | nd | | 1.22 | 0.02 | 0.07 |
| Spent biomass | — | — | — | — | |
| Fraction F1 | <0.1 | 0.18 | 45.4 | 1.1 | 1.90 |
| Fraction F2 | <0.1 | 0.24 | 25.87 | 1.05 | 2.42 |
| Fraction F3 | | 0.28 | 15.06 | 0.70 | 1.96 |
| Fraction F4 | | 0.29 | 8.46 | 0.37 | 1.13 |
| Fraction F5 | | 0.27 | 6.10 | 0.26 | 0.72 |

CBD extraction Recovery=90.4 wt %
Total cannabinoids recovery=92 wt %

### Examples 10 and 11

The following example refer to duplicate experiments (Expt. 1 and Expt. 2) carried out on two hemp types as shown (referred to as Examples 10 and 11). The experiments were carried out quantitatively in order to assess content of terpenes present in the extracted products. The extracts were collected in fractions but not all fractions were fully analysed.

### Biomass Type: L 2.1 (Example 10) Decarboxylated

| | | | Expt 2 |
|---|---|---|---|
| | Expt 1 | | (% in |
| Selected | (% in extract) | | extract) |
| component | Fraction 1 | Fraction 2 | Fraction 1 |
| B-caryophyllene | 2.73 | 0.94 | 2.94 |
| Humulene | 0.76 | negligible | 0.34 |
| α-Bisabolol | 3.0 | — | 0.34 |
| Limomene | 0.81 | — | 0.37 |
| Total terpenes % | 7.3 | 0.94 | 4.0 |
| CBD % | 41.51 | 55.2 | 44.7 |

### Biomass Type: L 1.7 (Example 11) Non-Decarboxylated

| | Expt 1 | Expt 2 |
|---|---|---|
| Selected | Fraction1 | Fraction1 |
| component | (% in extract) | (% in extract) |
| B-caryophyllene | 13.61 | 15.42 |
| Humulene | 4.23 | 4.77 |
| α-Bisabolol | 0.3 | 0.36 |
| Limomene | 1.22 | 0.13 |
| Total terpenes % | 19.36 | 20.68 |
| CBDA % | 11.1 | 13.3 |

In both examples 10 and 11 it was estimated that substantially the total amount of terpenes in the biomass was extracted in the process. The extracts described can be used in preparation of dietary ingredients, dietary supplements and/or health supplements.

As an alternative to use of the apparatus of FIG. 1, the apparatus described in FIG. 2 may be used.

Referring to FIG. 2, apparatus for carrying out fractional extraction using a liquefied gas as extraction medium comprises an extraction column 102 in which material to be extracted is tightly packed. The column may be jacketed and include heating/cooling means for temperature control. Upstream of the column 102 is a hold vessel 104 for containing the liquefied gas, for example HFC 134a. The vessel 104 is connected downstream, by pipework 106, to the upper end of the column 102 for transferring the extraction medium into the column 102. A liquid metering pump 108 is provided in pipework 106 for controlling the flow of extraction solvent to the column. Immediately upstream of the column, the pipework includes an in-line heat exchanger 110 arranged to heat (or cool) liquid prior to its passage into the column. Between the pump 108 and heat-exchanger 110, there is a modifier solvent supply pipe 112 which is arranged to deliver a modifier solvent from a storage vessel 114 into the pipework 106 so that it mixes with liquefied gas from vessel 104. A liquid metering pump 116 in supply pipe 112 controls the flow of liquid within the pipe 112.

Downstream of the vessel 102 are shown three collection/evaporation vessels 120, 122, 124 although more such vessels would generally be provided for collecting more than three different aliquots. Each of the vessels 120, 122, 124 includes an inlet pipe 126 and an outlet pipe 128 each having associated control valves 130. The vessels 120, 122, 124 are arranged to communicate with column 102 via pipeline 132 which is connected to the bottom of the column. A monitoring device 134 is arranged to monitor and/or analyse fluid flowing in pipeline 132. Downstream of pipeline 132 is a pipeline 136 which communicates with vessel 104 and includes an associated gas compressor 138 for liquefying gas prior to its passage back into the vessel 104.

The apparatus further includes any necessary in-line filters, one-way valves, flow control valves, pressure regulators and pressure release valves and instrumentation for reading temperature, pressure and pH to allow appropriate process control and safe operation of the apparatus.

In use, a vacuum pump (not shown) is operated to remove air from the apparatus after the material to be extracted has been packed into the column 102. Liquefied gas is then charged to the vessel 104 and co-solvent, if this is used, is charged into vessel 114. With any heating/cooling means of the apparatus appropriately set, liquid is passed from vessel 104 to the column 102. The liquid slowly percolates through the material in the column and extracts compounds from the material as it does so. Initially, the most soluble compounds included in the biomass are extracted preferentially and these are entrained with liquid as it passes from the column into pipeline 132. The liquid (and entrained extract) is then directed into vessel 120 by opening the appropriate valve. After a period of time which may be determined in dependent upon an output from monitoring device 134, subsequent liquid passing out of the column is directed into vessel 122. Subsequently, it is directed into vessel 124 and later to other vessels (if provided). Thus separate aliquots are collected in vessels 120, 122, 124 and the constitution of the extracts therein should differ, with compounds or compositions which are most soluble in liquid passing through the column being more concentrated in the vessels which initially are used for collection and less soluble compounds or compositions being more concentrated in collection vessels used later in the process.

The constitution of extracts may also be affected by delivering a co-solvent from vessel 114 into pipeline 108 and mixing the co-solvent with liquid from vessel 104. The combined extraction solvent may then be adapted to extract preferentially certain compounds or compositions. The co-solvent may be delivered as described herein for manipulating the extraction of the biomass. Additionally and/or alternatively, the heat-exchanger 10 may be used to adjust the temperature of the extraction solvent thereby to control the nature of compounds or compositions preferentially extracted. Also, the temperature of the column itself (and thereby the biomass therein) may be adjusted as another means of affecting the nature of extracts.

After the extraction of the biomass has been completed (or prior to completion whilst extraction in the column 102 is ongoing), the control valve to outlet pipe 128 of vessel 120 may be opened and compressor 138 operated to remove liquefied solvent from the vessel 120 and return it to vessel 104, leaving the compound(s)/composition(s) in vessel 20. This process may be repeated to isolate the different extracts in the respective vessels 120, 122, 124.

The aforementioned examples involve treatment of a cannabinoid-containing biomass which has been decarboxylated (as is conventional in industry) as described in Example 1. However, in some cases, it is found that such decarboxylation can result in charring of the plant material, a darkening in its colour and production of acrid smoke. This may also result in loss of cannabinoid content and production of lower purity extract. Example 8 which follows describes treatment of biomass prior to any decarboxylation.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of extracting at least one cannabinoid from a biomass, the method comprising the following steps:
(i) contacting the biomass with a solvent formulation which comprises a C1-4 fluorinated hydrocarbon or a C1-4 hydrofluorocarbon ether, thereby to charge the solvent formulation with an extract from the biomass; and
(ii) separating charged solvent formulation from the biomass.

2. A method according to claim 1, wherein said biomass comprises industrial hemp and/or industrial hemp which has been treated to derivatise one or more cannabinoids included in the hemp and wherein said biomass includes a THC content of less 0.3%.

3. (canceled)

4. A method according to claim 1, wherein said biomass comprises leaves and/or flowers which comprise one or more cannabinoids and/or derivatives of one or more cannabinoids, wherein the sum of the wt % of leaves and flowers in said biomass is at least 98 wt % and wherein said cannabinoid is naturally-occurring in the biomass or is a derivative of a cannabinoid which is naturally-occurring in the biomass.

5. A method according to claim 1, wherein said biomass includes CBD and/or CBDA, wherein a sum (S1) of the wt % of CBD and CBDA in the biomass on a dry matter basis is in the range 0.3 to 4 wt %; and wherein said biomass includes THC and/or THCA, wherein a sum (S2) of the wt % of THC and THCA in the biomass on a dry matter basis is less than 0.3 wt %.

6. (canceled)

7. A method according to claim 5, wherein the sum of sum (S1) and sum (S2) is in the range 0.3 to 4 wt % and/or a ratio defined as the sum (S1) divided by the sum (S2) is in the range 6 to 15.

8. (canceled)

9. A method according to claim 4, wherein in an embodiment (B), said biomass is treated prior to step (i) to derivatise a naturally-occurring cannabinoid in the biomass.

10. A method according to claim 1, wherein the ratio of the wt % of CBD divided by the wt % of CBDA in the biomass is greater than 10.

11. (canceled)

12. (canceled)

13. (canceled)

14. (canceled)

15. (canceled)

16. (canceled)

17. A method according to claim 1, wherein, in step (i), contact of biomass with solvent formulation takes place when the biomass is at a temperature range of less than −5° C. to 20° C.; and wherein the solvent formulation which contacts the biomass is at a temperature range of between 0° c. to 40° C.

18. A method according to claim 1, wherein said solvent formulation comprises the C1-4 fluorinated hydrocarbon which is non-chlorinated.

19. (canceled)

20. A method according to claim 1, wherein said solvent formulation comprises a solvent selected from: iodotrifluoromethane, 1,1,1,2,3,3,3-heptafluoropropane (HFC-227 EA), 1,1,1,2,2,3,3-heptafluoropropane (HFC-227CA) and 1,1,1,2-tetrafluoroethane (HFC-134a).

21. A method according to claim 1, wherein said solvent formulation consists essentially of 1,1,1,2-tetrafluoroethane.

22. (canceled)

23. (canceled)

24. (canceled)

25. A method according to claim 1, wherein the total weight of waxes in the charged solvent and/or an extract derived therefrom is less than the total weight of waxes in the biomass after the biomass has been treated in the method and/or after step (ii).

26. A method according to claim 1, wherein the wax ratio, defined as the total weight of waxes in the biomass after treatment in the method and/or after step (ii) divided by the total weight of waxes in the charged solvent is at least 5.

27. A method according to claim 1, wherein an extract produced after step (ii) after removal of said solvent formulation includes less than 0.5 wt % total waxes.

28. A method according to claim 1, wherein, in the extract and/or charged solvent formulation, the cannabinoid ratio, defined as the total weight of non-wax based cannabinoids divided by the total weight of waxes in the extract is at least 100.

29. A method according to claim 1, wherein an extract produced after step (ii) after removal of said solvent formulation is a mobile oil at 25° C. or a mixture of an oil and an amount of solid or crystalline material.

30. An extract from a biomass wherein said extract includes less than 0.05 wt % of waxes; wherein a cannabinoid ratio, defined as the total weight of non-wax based cannabinoids divided by the total weight of waxes in the extract is at least 5; wherein a ratio (A) defined as (the sum of the weights of CBD and CBDA in the extract):(the weight of terpenes in the extract) is in the range 2:1 to 25:1; wherein a ratio (B) defined as (the sum of the weights of CBD and CBDA in the extract):(the weight of mono and di-terpenes in the extract) is in the range 2:1 to 25:1; wherein a ratio (C) defined as (the sum of the weights of CBD and CBDA in the extract):(the sum of the weights of beta-caryophyllene, humulene, alpha-bisabolol, alpha-pinene, myrcene and limonene in the extract) is in the range 2:1 to 25:1; and wherein said extract includes at least 0.0001 wt % of HFC134a.

31. (canceled)

32. (canceled)

33. (canceled)

34. (canceled)

35. (canceled)

36. (canceled)

37. (canceled)

38. (canceled)

39. (canceled)

40. A method according to claim 2, wherein:
said biomass includes a THC content of less than 0.2%;
wherein said biomass includes CBD and/or CBDA, wherein a sum (S1) of the wt % of CBD and CBDA in the biomass on a dry matter basis is in the range 1.0 to 3.0 wt %;
wherein said biomass includes THC and/or THCA, wherein a sum (S2) of the wt % of THC and THCA in the biomass on a dry matter basis is less than 0.3 wt %;
wherein the sum of sum (S1) and sum (S2) is in the range 0.3 to 4 wt %;
wherein a ratio defined as the sum (S1) divided by the sum (S2) is in the range 6 to 15.

41. A method according to claim 2, wherein:
the ratio of the wt % of CBD divided by the wt % of CBDA in the biomass is greater than 10;
said solvent formulation consists essentially of, 1,1,1,2-tetrafluoroethane;
a wax ratio, defined as the total weight of waxes in the biomass after treatment in the method and/or after step (ii) divided by the total weight of waxes in the charged solvent, is at least 40;
an extract produced after step (ii) after removal of said solvent formulation includes less than 0.05 wt % total waxes;
in the extract and/or charged solvent formulation, the cannabinoid ratio, defined as the total weight of non-wax based cannabinoids divided by the total weight of waxes in the extract is at least 100.

42. A method according to claim 40, wherein:
the ratio of the wt % of CBD divided by the wt % of CBDA in the biomass is greater than 10;
said solvent formulation consists essentially of, 1,1,1,2-tetrafluoroethane;
a wax ratio, defined as the total weight of waxes in the biomass after treatment in the method and/or after step (ii) divided by the total weight of waxes in the charged solvent, is at least 40;
an extract produced after step (ii) after removal of said solvent formulation includes less than 0.05 wt % total waxes;
in the extract and/or charged solvent formulation, the cannabinoid ratio, defined as the total weight of non-wax based cannabinoids divided by the total weight of waxes in the extract is at least 100.
